(19) United States
(12) Patent Application Publication
(10) Pub. No.: US 2013/0209953 A1
(43) Pub. Date: Aug. 15, 2013

(54) SYSTEM AND METHOD FOR GUIDING DENTAL TREATMENT TOOLS

(71) Applicants: David Arlinsky, Attlit (IL); Gershon Goldenberg, Carcur (IL); Pinchas Kfir, Petach Tikva (IL); David Arlinsky, Atlit (IL)

(72) Inventors: David Arlinsky, Attlit (IL); Gershon Goldenberg, Carcur (IL); Pinchas Kfir, Petach Tikva (IL); David Arlinsky, Atlit (IL)

(73) Assignee: IGS DENTAL LTD., Kiryat Shmona (IL)

(21) Appl. No.: 13/636,891

(22) PCT Filed: Mar. 28, 2011
(86) PCT No.: PCT/IL11/00279

§ 371 Date: May 2, 2013

Related U.S. Application Data
(60) Provisional application No. 61/318,321, filed on Mar. 28, 2010.

(51) Int. Cl. A61C 1/08; A61B 6/14; A61C 1/00; A61C 3/02; A61C 8/00; A61C 19/04; A61B 6/03

(52) U.S. Cl. CPC A61C 1/082; A61C 19/04; A61B 6/145; A61B 6/032; A61C 3/02; A61C 8/009; A61C 1/0046
USPC 433/27; 433/215

(57) ABSTRACT

The invention is a system and method for guiding a dental treatment tool. The system comprises a guidance device inserted into the mouth and an analyzer unit receiving tool location information from the device and providing an image indicating the location of the tool in relation to the jaw. The guidance device provides a platform to which any type of dental tool can be attached and supports the tool, allowing it to be moved in any direction. The system tracks the motion of the tool and displays its location and orientation relative to a three dimensional coordinate system whose origin is located on part of the device. Prior to the procedure the practitioner designs a surgery or treatment path, which is displayed on top of an image of a region in the mouth. An image of the tool showing its instantaneous location and orientation is displayed as an overlay.

### FIELD OF THE INVENTION

The invention is related to the field of medical devices. Specifically the invention relates to the field of systems for assisting a practitioner to guide dental treatment tools.

### BACKGROUND OF THE INVENTION

Many different types of procedures are performed by dentists and dental surgeons using hand-held and hand-guided tools, e.g. dental drills, files, dental implants attached to a dental handpiece, grinding wheels, and lasers, on the teeth, facial bones, and jaws of patients. These procedures require skill and much care to prevent collateral damage to surrounding anatomical structures. The procedures are often difficult to carry out because of the limited volume of space within the mouth of the patient that restricts the ability of the practitioner to align or move the tool as he would like during the course of the procedure. Frequently the operating site is difficult to reach or to see and other factors such as unexpected movement by the patient can affect the orientation of the tool with relation to the desired direction.

The insertion of dental implants is an example of a procedure that illustrates the problem. Implants are widely used in the western world. A dental implant is inserted into a hole that is drilled in a jaw bone of a patient. The bone includes various anatomical structures (such as nerves, blood vessels, maxillary sinus etc.) that can be damaged due to the drilling process. The bone walls can also be damaged if the drilling process isn't performed in the accurate direction.

The risk of damaging these anatomical structures can be reduced by restricting the drilling zone, i.e. by very carefully planning and following the path that the drill should follow. As mentioned this is frequently much easier said then done with hand held and guided tools. Systems that have been proposed to aid the surgeon are based on real time tracking during the drilling process using multiple camera based tracking systems. In addition there have been proposed robotic systems in which the tool is gripped by a robotic arm that guides it according to a preplanned protocol or by the surgeon. Both the multiple camera and robotic systems are very expensive and difficult to use; therefore they are not in widespread use in the profession.

In general there is a growing need to be able to provide systems that will reduce the risks associated with procedures carried out using dental treatment tools. These systems must provide the surgeon with real time information that enables him to accurately guide his tool. In order to gain widespread acceptance the system must be ergonomically designed to fit within the mouth of the patient and allow the surgeon to manipulate the tool as closely as possible to the manner in which he is accustomed. Additionally the system should be made available at affordable price.

It is a purpose of the present invention to provide a system and method for guiding dental treatment tools that reduces the above mentioned risks.

It is another purpose of the present invention to provide an ergonomically designed system for guiding dental treatment tools that allows the surgeon to manipulate the tool as closely as possible to the manner in which he is accustomed.

Further purposes and advantages of this invention will appear as the description proceeds.

### SUMMARY OF THE INVENTION

In a first aspect the invention is a system for aiding a practitioner to guide a dental treatment tool by tracking the motion of the tool and displaying its location and orientation relative to a three dimensional coordinate system whose origin is located at a fixed location inside the mouth of a patient. The system comprises:
A) a guidance device comprising:
   i) a fixation unit adapted to be attached to and detached from the jaw and/or teeth of the patient;
   ii) an interfacing unit attached to the fixation unit in such a manner that allows the interfacing unit to be moved laterally relative to the fixation unit and to be tilted relative to a vertical axis, the interfacing unit being adapted to enable the dental treatment tool to be mechanically fixedly attached to it;
   iii) a sensor unit comprising several sensors adapted to produce signals related to the instantaneous location and orientation of the interfacing unit and attached tool relative to the fixation unit placed at different locations on the guidance device and a transmitter adapted to transmit the signals; and
B) an analyzer unit comprising:
   i) a receiver adapted to receive the signals transmitted by the transmitter in the guidance device;
   ii) a computing device comprising software adapted to determine the instantaneous location and orientation of the tool from the signals received by the receiver and other information; and
   iii) an output device adapted to present information relating to the location and orientation of the tool to the practitioner.

Using the system of the invention the motion of the interfacing unit and attached tool relative to the fixation unit is caused by the practitioner moving the tool.

The output device is adapted to visually display at least one of the following displayed as an overlay on top of an image of the jaw and teeth of the patient:
i) information representative of the surgical procedure and the boundaries of the zone within which it is safe to use the tool as determined by the practitioner prior to the procedure; and
ii) a template showing the instantaneous location and orientation of the tool.

In embodiments of the system of the invention the output device is adapted to warn the practitioner that the tool has reached the outer boundary of the safe zone as determined by him prior to the procedure or if there is an indication that continued use of the tool might cause damage to the patient in at least one of the following ways:
i) by emitting an audible signal;
ii) by displaying a prominent visible sign or message on top of all other images on a display device; and
iii) by automatically preventing the tool from operating.

In embodiments of the system of the invention the interfacing unit is attached to the fixation unit by means of an extension arm. The extension arm is adapted to allow the practitioner to move the interfacing unit and attached tool laterally along the jaw of the patient and to use the tool to perform procedures at more than one location in the jaw or facial bones without having to move the fixation unit. In these embodiments, after a dental treatment tool is mechanically attached to the interfacing unit, the interfacing unit can be moved along the extension arm until the distal tip of the dental treatment tool is over a surface that is in contact with a sensor. The dental treatment tool can then be lowered until the distal tip contacts the surface. This causes the sensor to generate a signal, which is transmitted to a computing device adapted to determine the length of the dental treatment tool from the signal.

In a second aspect the invention is a method of aiding a practitioner to guide a dental treatment tool. The method comprises the steps of:
a) obtaining images of the area in the mouth of a patient where a procedure requiring the use of the tool is to be carried out;
b) designing a desired surgery or treatment path and providing path information to a computing device of an analyzer unit;
c) inserting a guidance device comprising an interfacing unit attached to a fixation unit in such a manner that allows the interfacing unit to be moved laterally relative to the fixation unit and to be tilted relative to a vertical axis;
d) connecting the fixation unit to the jaw and/or teeth of the patient;
e) attaching the tool to the interfacing unit such that movement of the tool by the practitioner causes a corresponding movement of the interfacing unit relative to the fixation unit;
f) activating the tool while moving the tool according to the surgery or treatment path designed in step b;
g) sensing, by means of sensors placed at different locations on the guidance device, the location and the orientation of the of the interfacing unit and attached tool relative to the fixation unit and transferring signals indicative of the location and orientation to a transmitter;
h) transmitting the location and orientation signals to a receiver that is a part of an analyzer unit;
i) activating a computing device in the analyzer unit, the computing device comprising software adapted to determine the instantaneous location and orientation of the tool based on the signals received by the receiver; and
j) activating a display device in the analyzer unit to display information relating to the location and orientation of the tool to the practitioner.

In embodiments of the method of the invention steps f to j are carried out simultaneously in real-time.

Embodiments of the method of the invention may comprise an introductory step of inserting one or more positioning elements in a jaw of the patient. In embodiments of the method of the invention at least one of the positioning elements is adapted to connect a fixation unit to a jaw and the method comprises the step of connecting a fixation unit to the elements.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically illustrates a system according to an embodiment of the invention;

FIG. 2 schematically illustrates portions of a drilling tool, a jaw of a person, a fixation unit, and portions of an interfacing unit, according to an embodiment of the invention;

FIG. 3 schematically illustrates portions of the drilling tool, interfacing unit and jaw 12 according to an embodiment of the invention;

FIG. 4 schematically illustrates the main components of a guidance device according to an embodiment of the invention;

FIG. 5 schematically illustrates a synthetic image that includes a CT image, a drilling tool icon indicative of a position of the drill bit and path information indicative of a desired drilling path according to an embodiment of the invention; and

FIG. 6 schematically illustrates a method according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The method of the invention involves three basic steps: 1) taking a CT image of the region of the mouth including the area of the jaw, facial bone, or teeth at which the procedure is to be performed; 2) preparing a plan for carrying out the procedure and overlaying the plan on top of the CT image; 3) performing the procedure using the system of the invention which displays in real time an image representing the exact location and orientation of the tool treatment tool on top of the plan and CT image.

The system of the invention comprises a guidance device that is placed into the mouth of the patient. The guidance device consists of a fixation unit that is adapted to be fixed to the jaw (or teeth) near the desired location of the procedure. An interfacing unit interfaces between a dental treatment tool and the fixation unit. The interfacing unit is connected to the fixation unit in a manner that facilitates movement of the tool in relation to the fixation unit. The tool is fixedly attached to the interfacing unit such that movement of the tool by the practitioner causes a corresponding movement of the interfacing unit relative to the fixation unit. The connection between the interfacing unit and the fixation unit is adapted such that the surgeon can grip the tool in the normal manner and move it in any manner that he wishes. The guidance device is provided with sensors that measure the linear and angular motion of the interfacing unit with respect to the fixation unit in three dimensions. The information from the sensors is transmitted to an analyzer unit that comprises a computer comprising software adapted to use the information from the sensors, data related to the physical dimensions of the parts of the guidance device and the tool, information about the mechanical connection between the tool and the interface unit, and knowledge of the location of the fixation unit in the mouth to determine the exact location and orientation of the tool and a part of the tool, e.g. its distal end, in relation to the anatomical features.

In general the guidance device of the invention provides a platform to which any type of dental tool can be attached. The guidance device provides a platform that supports the tool and can be moved in any direction inside the mouth of a patient by the surgeon. It is emphasized that all of the motion of the tool is determined and guided by the surgeon. The role of the system of the invention is to track the motion of the tool and to display its location and orientation relative to a three dimensional coordinate system whose origin is located on a part of the guidance device that is fixed to the jaw of the patient. Thus any motion of the patient's head or jaw during the procedure does not have to be compensated for and does not affect the information that is presented to the surgeon.

For brevity of explanation and in order to illustrate the invention by means of a concrete example, a part of the following description refers to drilling a hole in which an implant is to be inserted in a jaw of a person. Those of skill in the art will appreciate that the system and method described below can be applied mutatis mutandis to procedures requiring the use of other dental treatment tools and also for non-dental medical procedures in other regions of a person or animal.

A system is provided. The system of the invention comprises: (i) a guidance device that is inserted into the mouth, and (ii) an analyzer unit that receives tool location information from the device and provides a synthetic image that indicates the location of the tool in relation to the jaw of the person. The synthetic image illustrates the region of interest, e.g. the region in which the implant should be inserted and its vicinity.

According to an embodiment of the invention the guidance device comprises a fixation unit, an interfacing unit, a sensor unit, and an information transmitting device, e.g. a wireless transmitter. The analyzer comprises a receiving device, a display device, and a computer.

According to an embodiment of the invention the fixation unit is adapted to be fixed to the jaw (or teeth) near the desired location of the procedure. The interfacing unit interfaces between a dental tool, e.g. a drilling tool, an implant insertion tool, a screw insertion tool, a file, a grinder, or a laser, and the fixation unit. The interfacing unit is connected to the fixation unit in a manner that facilitates movement of the drilling tool in relation to the fixation unit. The drill is fixedly attached to the interfacing unit such that movement of the drill by the surgeon causes a corresponding movement of the interfacing unit relative to the fixation unit.

In order to reduce the labor and expense of sterilization between procedures some or all of the components of guidance device can be disposable.

The interfacing unit and sensor unit are located very close to the drilling tool and thus can very accurately track the location of the drilling tool.

The system is very compact allowing the system to be installed in relatively small rooms; that is the system is designed to be used in private dentist offices and clinics and not necessarily in large operating theaters. The guidance device fits into the mouth of the patient and, if wireless communication is employed, the analyzer unit can be located in any desired location within a reception range of the wireless transmitter thus greatly simplifying the installation of the system.

The sensor unit is comprised of several sensors that are placed at different locations on the guidance device. The individual sensors are adapted to sense features of the interfacing unit that are related to its instantaneous location and orientation of the tool that moves when the tool is manipulated by the surgeon. The sensors are also adapted to send location and orientation signals to a transmitter that transmits the location and orientation signals to a receiver of the analyzer unit. Communication between the sensors and the analyzer unit can be via wired or wireless communication.

The analyzer, comprises computing means, e.g. a PC, that is equipped with software adapted to determine the location and orientation of the interfacing unit and from this the location and orientation of the tool, e.g. a drill bit, which is rigidly attached to the interfacing unit. The determination is based upon other things on the instantaneous values of the location and orientation signals received from the sensors. The analyzer unit also comprises output means, e.g. a display screen, to output information showing the location and orientation of the tool to a surgeon.

According to an embodiment of the invention the analyzer unit displays the location and orientation information as an overlay on top of an image (2D or 3D) of the jaw and teeth of the person. The image is acquired by any method known in the art, e.g. x-ray imaging or computerized tomography (CT).

According to another embodiment of the invention the analyzer unit displays the boundaries within which the surgeon can safely work with the tool without damaging the surrounding anatomical structures. For example the displayed information can be path information representative of a desired drilling path as determined by the surgeon prior to the procedure. This type of information is displayed as an overlay on top of the image of the jaw and teeth of the person. In embodiments of the invention the instantaneous location and orientation of the drilling tool or the implant are displayed in addition to the path information.

In order to identify the area where the drilling or any other procedure is to take place, according to an embodiment of the invention, the acquisition of images (such as CT images) of the teeth and jaw of the person is preceded by inserting one or more positioning elements, e.g. screws, in the jaw of the person. These positioning elements may be made of radio-opaque material that is visible in the images of the teeth and jaw such as stainless steel, titanium, barium painted plastic material to provide reference points. They may be smaller than the implant. These positioning elements are used when planning the implant surgery and generating path information. In embodiments of the invention the positioning elements are designed and located such that the fixation unit can be detachably connected to at least one of them, thus increasing the stability of the fixation unit and providing a highly accurate positioning of the fixation unit.

The positioning elements are needed in cases wherein there are very few or no teeth in the jaw of the patient; however, in many instances, the teeth themselves are easily identified in x-ray or CT scans and therefore there is no necessity of inserting the positioning elements. In order to enable features of the teeth to be used as reference points in the images the fixation unit is designed to be affixed to the teeth or jaw in a manner that leaves the relevant parts of the teeth exposed.

Another aspect of the invention is the provision of methods of using the system of the invention to perform a surgical procedure. The steps of the procedure obviously are dependent on its exact nature and are hard to describe for a general case. Therefore a method for providing a hole for inserting a dental implant will be illustrated. It is anticipated that persons of the art will encounter no difficulties in adapting the illustrative method mutatis mutandis for use with any type of surgical tool, dental tool, or procedure.

Several different embodiments of an implantation method, each of which may include at least some of the following stages or combinations thereof, are possible: (i) if necessary, inserting one or more positioning elements in the jaw of the person, the one or more elements provide reference points and may be used to connect a fixation unit; (ii) obtaining images of the jaw and teeth of a person; (iii) designing a desired implant surgery path and providing path information; (iv) connecting a fixation unit of a guidance device to the jaw or teeth or to at least one of the positioning elements; (v) detachably connecting a drilling tool to an interfacing unit of the guidance device that is connected to the fixation unit, wherein the drilling tool is attached to the interface unit so that when the drilling tool is moved by the surgeon the interface unit moves with it relative to the fixation unit; (vi) drilling a hole for an implant while moving the drilling tool, (vii) sensing, by means of sensors, collectively known as a sensing unit, positioned at several of locations on the interfacing unit; wherein the sensors are adapted to produce signals related to the movements of the interfacing unit and drilling tool that is rigidly, mechanically attached to the interfacing unit; (viii) transmitting the signals from the sensors to an analyzer unit; (ix) analyzing by means of software in the computing device of the analyzing unit the signals to determine the instantaneous location of the drilling tool or more specifically the drill bit, from the signals and information related to dimensions and the geometric relationship between components of the guidance device, the drilling tool, and the mechanical connection of the drilling tool to the interfacing unit; (x) displaying information indicative of the location and orientation of the drilling tool to a surgeon; and (xi) using the displayed information to guide the drill bit along the desired implant surgery path until the drilling of the hole is completed.

Stages (vi) to (xi) may be executed in parallel and in real time. Thus a surgeon can receive real time feedback to assist him in managing the drilling process accordingly.

The above mentioned method and system can be adapted to track movement of the drilling tool with sub-millimetric precision and thereby facilitate a highly accurate and safe drilling process. The method and system of the invention allow the diameter of the drilling zone to be increased and its boundaries may be within a fraction of a millimeter from anatomical structures of the person.

The following figures illustrate a portion the system, portions of the device and a jaw of a person according to an embodiment of the invention. It is noted that these figures provide only a few possible embodiments while other equivalent embodiments can also be within the scope of the claims.

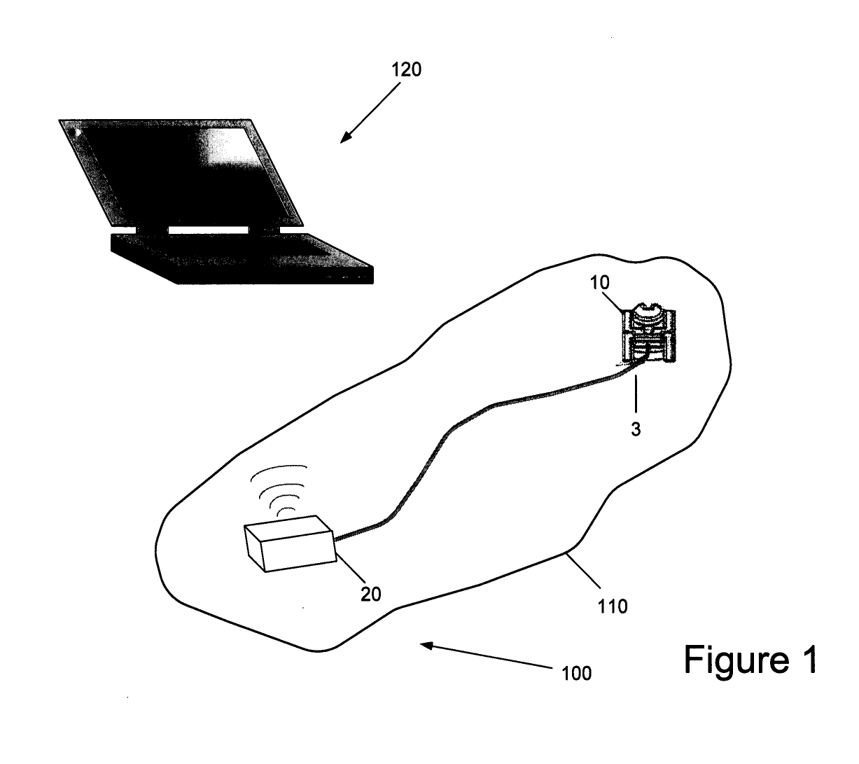

FIG. 1 illustrates system 100 according to an embodiment of the invention. System 100 includes analyzer unit 120 and guidance device 110.

In this embodiment guidance device 110 includes a wireless transmitter 20, sensor unit 3, fixation unit 10, and an interfacing unit (not shown in FIG. 1) that comprises sensor unit 3 and is moveably attached to fixation unit 10.

The wireless transmitter 20 receives signals indicative of the location and orientation of a tool attached to the interfacing unit relative to the fixation unit 10 from sensors in sensor unit 3 and transmits them to the analyzer unit 120.

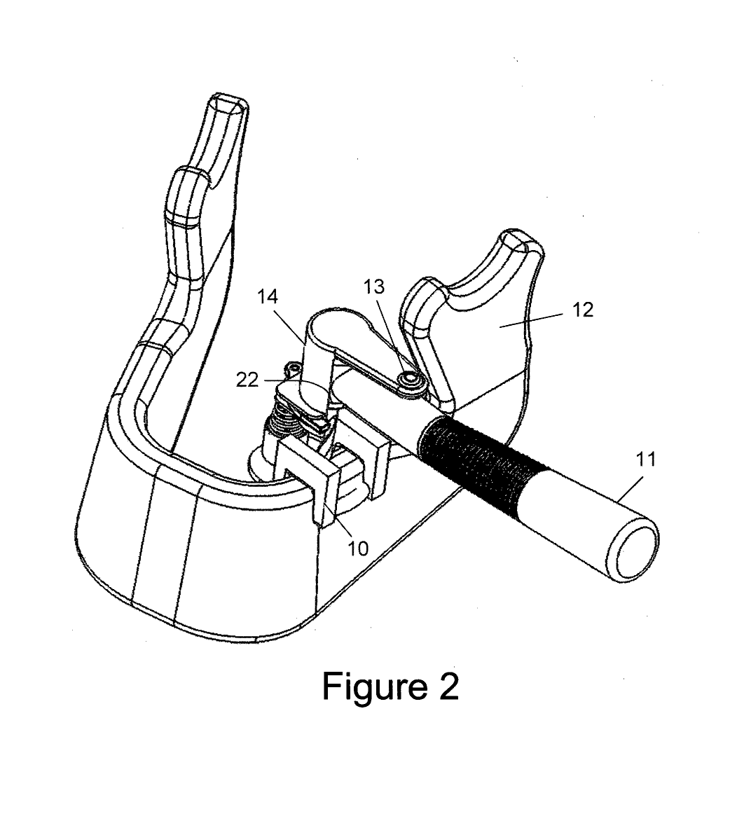

FIG. 2 illustrates portions 11 and 22 of a drilling tool, a jaw 12 of a person, fixation unit 10, and portions 14 and 13 of an interfacing unit, according to an embodiment of the invention.

Fixation unit 10 is fixed to the teeth (not shown in FIG. 2) of the person near a location that should be drilled by the drilling tool. Fixation unit 10 includes two bridge shaped elements that are parallel to each other. In other embodiments the fixation unit may have a different shape that is adapted to allow it to be attached to the bone that is to be drilled.

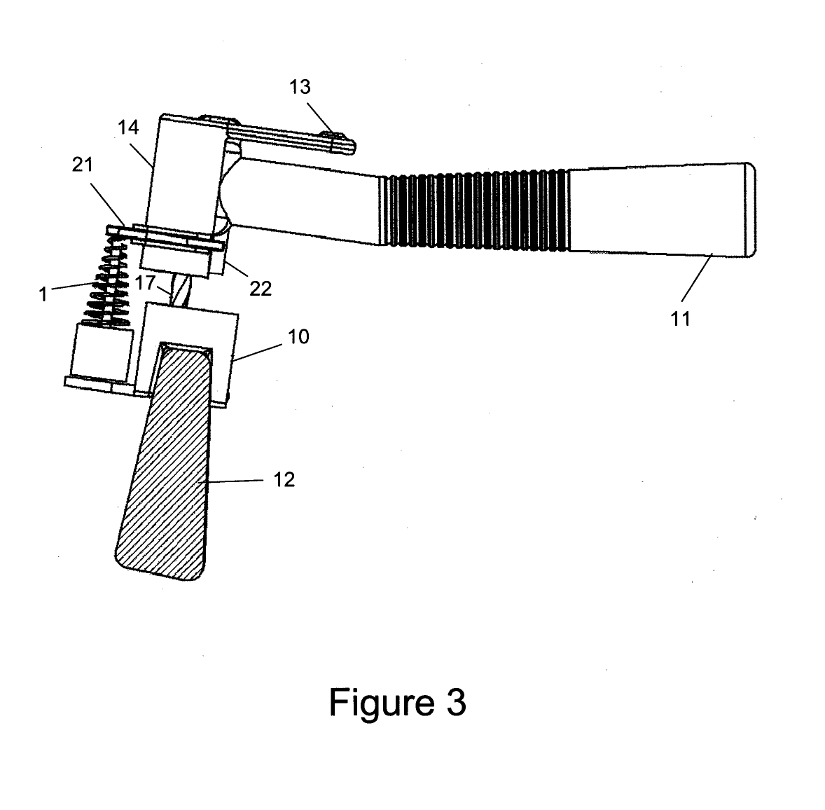

The drilling tool includes a drill bit (denoted 17 in FIG. 3), that is detachably connected to a vertical cylindrical portion 22 of the drilling tool. The body of the drilling tool is formed of the vertical cylindrical portion 22 and of a horizontal cylindrical portion 11. Horizontal cylindrical portion 11 functions as a handle by means of which the surgeon can manually move the interfacing unit laterally along the jaw, can cause the interfacing unit to pivot about a vertical axis, and can move the interfacing unit up and down relative to a horizontal axis.

The interfacing unit has a housing 14 that is shaped to match a front end of the drilling tool so that the drilling tool may be firmly connected to the interfacing unit during the drilling process to prevent movement of the drilling tool relative to the interfacing unit.

FIG. 2 illustrates a button 13 that symbolically represents the mechanism for mechanically coupling the drilling tool to the interfacing unit. Button 13 is pressed to release the drilling tool when the drilling process is completed. It is noted that other components that are not shown may be used in order to fasten the drilling tool the interfacing unit.

FIG. 3 illustrates portions of the drilling tool, drill bit 17, interfacing unit, and fixation unit 10 of the guidance device according to an embodiment of the invention attached to the jaw 12 of a patient.

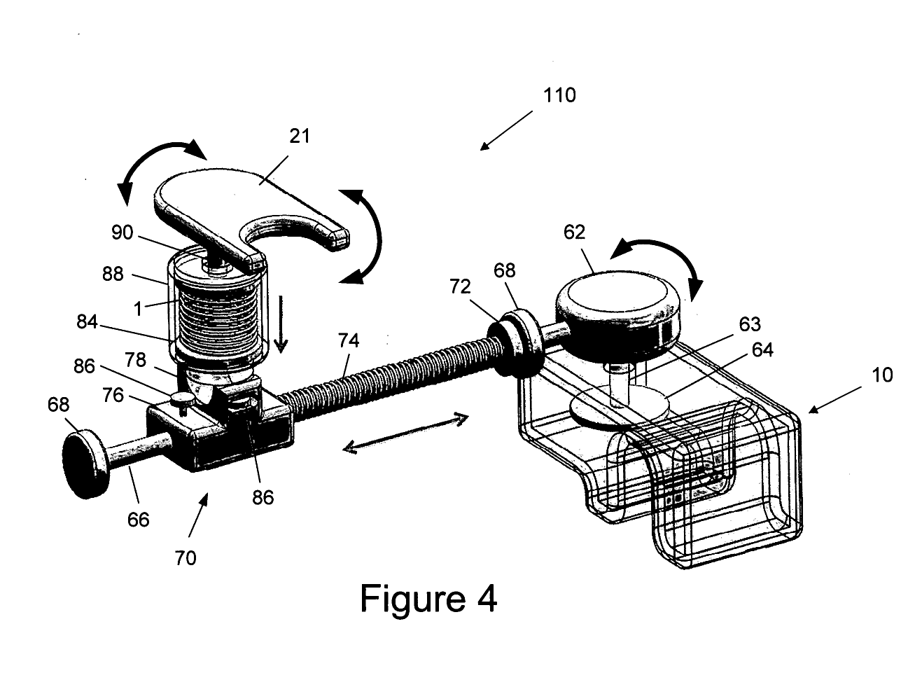

FIG. 4 illustrates the guidance device 110 according to an embodiment of the invention. In this figure are shown the main components of the sensing unit and interfacing unit 70 that play a role in measuring the location and orientation of the drilling tool (bit 17) in respect to the fixation unit 10.

Fixation unit 10 is adapted to be attached to the jaw or teeth at a predetermined position. Interface unit 70 comprises a base 76 that fits slidingly on an extension arm 66 that is rigidly attached to extension arm attachment 62. Extension arm attachment 62 is rigidly attached to the upper end of vertical axle 63 that is attached at its lower end to fixation unit 10 in such a way that allows axel 63 to be turned freely with respect to fixation unit 10 in the direction indicated by the wide double headed arrow, At the base of axle 63 is located angle encoder 64 that measures the angle of rotation of axle 63 and everything attached to it relative to the fixation unit.

At the two ends of extension arm 66 are fixed stoppers 68 to limit the lateral motion of the interface unit base 76. Between interface unit base 76 and the stopper 68 closest to the fixation unit 10 a spring 74 is fitted over extension arm 66. As interface unit base 76 moves back and forth along extension arm 74, the length of the spring 74 changes. A force sensor 72 at the end of spring 74 measures the force exerted on it caused by compression and decompression of spring 74. The force measured by sensor 72 can be directly converted into the distance of interface unit base 76 from stopper 72 or the axis of axle 63. The purpose of extension arm 66 is to allow the surgeon to move the drill laterally along the jaw. An advantage of the extension arm is that it enables the surgeon to drill a number of holes in the jaw without having to move the fixation unit 10.

The main components of the interface unit 70 are shown in FIG. 4. Horizontal plate 21 is adapted to allow a tool, e.g. a drilling tool, to be fixedly attached to it. Horizontal plate 21 is rigidly attached to the top of post 90, which can be moved up and down compressing and decompressing a spring 1 inside a cylindrical housing 88. At the bottom of spring 1 is a force sensor 84 that measures the amount of compression of spring 1 from which the height of horizontal plate 21 relative to interface unit base 76 can be determined. The lower surface of cylindrical housing 88 is attached fixedly to spherical contact bearing 78. Spherical contact bearing 78 is held in interface unit base 76 so that it can pivot in all directions about a fixed contact point. Two angle encoders 86 measure the angular motion of spherical contact bearing 78 and thus of post 90, horizontal plate 21 and the attached drilling device.

All of the rotational and lateral movement of the device is caused by the surgeon who simply grips the treatment instrument and moves it as he normally would in the absence of the device of the invention. Signals from the force sensors and angle encoders are transmitted to the computer of the analyzer unit, which contains information related to the dimensions and other parameters of all of the critical components of the device, e.g. coefficients of compression of the springs, length of extension arm 62, and the maximum height of horizontal plate 21 above interface unit base 76. The computer comprises software adapted to use these dimensions and parameters as well as the signals from the encoders and sensors to accurately determine the location of and orientation of the tool relative to the known location of fixation unit 10 and to graphically display this location as an overlay on a CT image.

In practice most surgeons change drill bits at least once during a drilling procedure, for example a small diameter bit is used to drill a guide hole that is then widened by using at least one more bits having a larger diameter. Since each of these bits, in addition to having different diameters normally have different lengths, recalibration of the system would be needed. Embodiments of the device of the invention are adapted to easily accomplish this calibration. In these methods each time a bit is changed the drill is pushed along the extension arm and pushed downward until the tip and jaw 12 of the bit just contacts the top of extension arm attachment 62. The signals from force sensor 84 can then be converted by the computer of the analyzer unit to determine the relative lengths of the drill bits.

The interfacing unit 70, fixation unit 10 or at least parts of them may be disposable; thus they are replaced for each implant surgery.

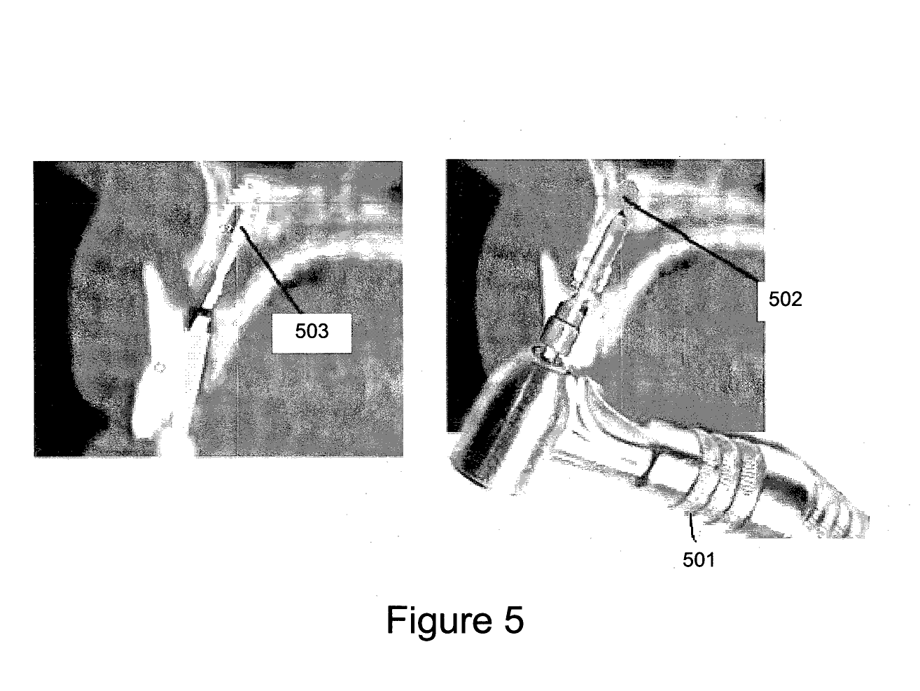

FIG. 5 illustrates typical synthetic images that could be displayed for a surgeon during a drilling procedure. The right side synthetic image shows a drilling tool 501 and a drilling path 502 (hole) overlaid on a CT image. The synthetic image on the left illustrates an implant 503 inserted in the hole after the completion of the drilling.

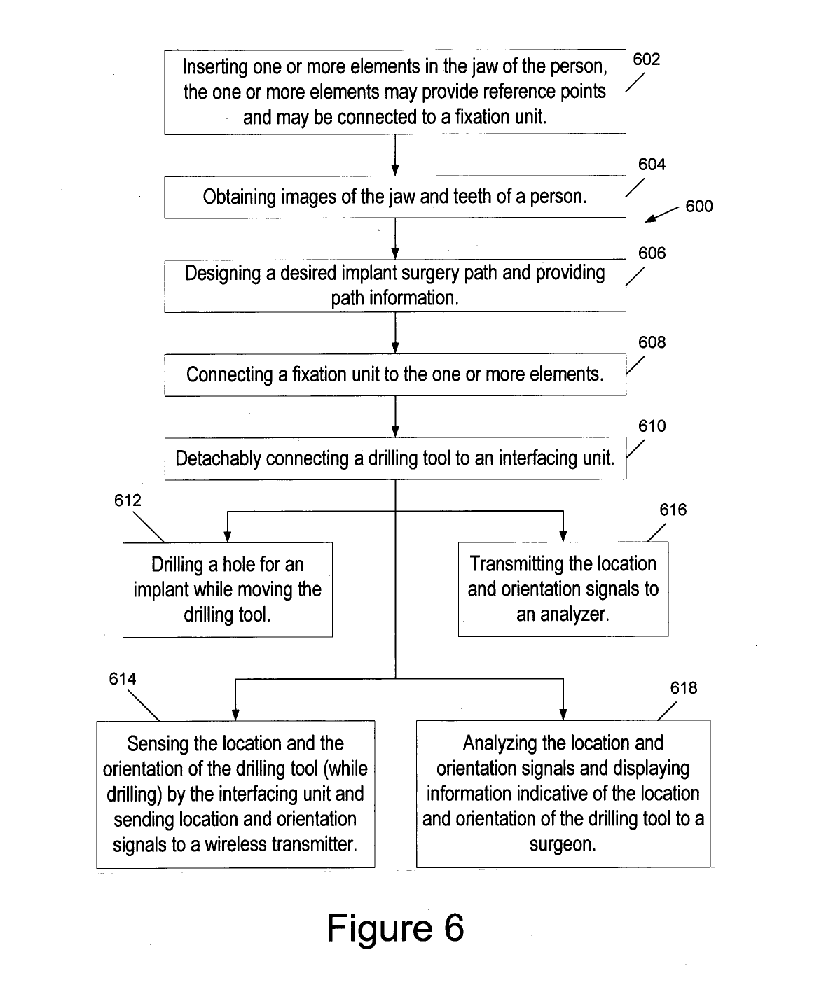

FIG. 6 is a flow diagram showing the steps of a method 600 for drilling a hole in the jaw of a patient according to an embodiment of the invention. The specific reference to a drilling tool and drilling procedure in the description of method 600 is meant to illustrative the method by means of a concrete example. However it is to be recognized that method 600 is a generic method and some or all of the steps can be modified or eliminated mutatis mutandis for use with different tools or procedures without departing from its general framework.

Method 600 starts at stage 602 with inserting one or more positioning elements in the bone, e.g. jaw, of the person. The one or more elements provide reference points and may be adapted to connect a fixation unit to the bone.

Stage 602 is followed by stage 604, which consists of obtaining images, e.g. CT images, of the area in the bone where the drilling is to be carried out. For example in the case of a procedure for installing a dental implant the images would include a portion of the jaw and teeth of a person.

Stage 604 is followed by stage 606 comprising designing a desired surgery path and providing path information to a computer of an analyzer unit.

Stage 606 is followed by stage 608 comprising connecting the fixation unit to the bone, e.g. the jaw and/or teeth, and/or to at least one of the one or more elements installed in stage 602.

Stage 608 is followed by stage 610 comprising detachably connecting a drilling tool to an interfacing unit that is connected to the fixation unit.

Stage 610 is followed by stages 612, 614, 616 and 618, which are carried out simultaneously in real-time.

Stage 612 comprises drilling a hole while the surgeon moves the drilling tool according to the surgery path that he designed in stage 606.

Stage 614 comprises sensing, by means of the sensing elements of a sensor unit, the location and the orientation of the drilling tool (while drilling) and transferring signals indicative of the location and orientation of the drill bit to a transmitter.

Stage 616 comprises transmitting the location and orientation signals to a receiver that is a part of an analyzer unit. The transmission can be wireless or wired and the transmitter in the sensing unit and receiver in the analyzer unit are suitably adapted for type of transmission used in a specific embodiment.

Stage 618 comprises analyzing the location and orientation signals by means of software in a computer of the analyzer unit to convert the signals to information indicative of the location and orientation of the drilling tool.

Stage 618 may comprise displaying the information to a surgeon on a computer screen, a HUD (Heads Up Display), or on special eyeglasses that can be worn by the surgeon.

Stage 618 may comprise generating an alert (visual/audio or both) and/or automatically stopping the drilling process (by transmitting a control signal to a drilling tool module) if the drill bit reaches the outer boundary of the drilling zone as determined in stage 606 or if there is an indication that continued drilling might cause damage to the patient.

Stage 612 of drilling the hole can be controlled by feedback provided during stage 618. Thus a dental surgeon/surgeon may determine the current location and orientation of the drilling based on the audio or visual alert signals.

While certain features of the invention have been illustrated and described herein, many modifications, substitutions, changes, and equivalents will now occur to those of ordinary skill in the art. It is, therefore, to be understood that the invention may be carried out with many variations, modifications, and adaptations, without exceeding the scope of the claims.

1. A system for aiding a practitioner to guide a dental treatment tool by tracking the motion of said tool and displaying its location and orientation relative to a three dimensional coordinate system whose origin is located at a fixed location inside the mouth of a patient, said system comprising:
A) a guidance device comprising:
i) a fixation unit adapted to be attached to and detached from the jaw and/or teeth of said patient;
ii) an interfacing unit attached to said fixation unit in such a manner that allows said interfacing unit to be moved laterally relative to said fixation unit and to be tilted relative to a vertical axis, said interfacing unit being adapted to enable said dental treatment tool to be mechanically fixedly attached to it;
iii) a sensor unit comprising several sensors adapted to produce signals related to the instantaneous location and orientation of said interfacing unit and attached tool relative to said fixation unit placed at different locations on said guidance device and a transmitter adapted to transmit said signals; and
B) an analyzer unit comprising:
i) a receiver adapted to receive said signals transmitted by said transmitter in said guidance device;
ii) a computing device comprising software adapted to determine the instantaneous location and orientation of said tool from said signals received by said receiver and other information,; and
iii) an output device adapted to present information relating to the location and orientation of said tool to said practitioner.

2. The system of claim 1, wherein the motion of the interfacing unit and attached tool relative to said fixation unit is caused by the practitioner moving the tool.

3. The system of claim 1, wherein the output device is adapted to visually display at least one of the following displayed as an overlay on top of an image of the jaw and teeth of the patient:
i) information representative of the surgical procedure and the boundaries of the zone within which it is safe to use the tool as determined by the practitioner prior to the procedure; and
ii) a template showing the instantaneous location and orientation of said tool.

4. The system of claim 1, wherein the output device is adapted to warn the practitioner that the tool has reached the outer boundary of the safe zone as determined by him prior to the procedure or if there is an indication that continued use of said tool might cause damage to the patient in at least one of the following ways:
i) by emitting an audible signal;
ii) by displaying a prominent visible sign or message on top of all other images on a display device; and
iii) by automatically preventing said tool from operating.

5. The system of claim 1, wherein the interfacing unit is attached to the fixation unit by means of an extension arm that is adapted to allow the practitioner to move said interfacing unit and attached tool laterally along the jaw of the patient and to use said tool to perform procedures at more than one location in the jaw or facial bones without having to move said fixation unit.

6. The system of claim 5, wherein, after a dental treatment tool is mechanically attached to the interfacing unit, said interfacing unit can be moved along the extension arm until the distal tip of said dental treatment tool is over a surface that is in contact with a sensor; whereupon, said dental treatment tool is lowered until said distal tip contacts said surface, causing said sensor to generate a signal which is transmitted to a computing device adapted to determine the length of said dental treatment tool from said signal.

7. A method of aiding a practitioner to guide a dental treatment tool said method comprising the steps of:
a) obtaining images of the area in the mouth of a patient where a procedure requiring the use of said tool is to be carried out;
b) designing a desired surgery or treatment path and providing path information to a computing device of an analyzer unit;
c) inserting a guidance device comprising an interfacing unit attached to a fixation unit in such a manner that allows said interfacing unit to be moved laterally relative to said fixation unit and to be tilted relative to a vertical axis;
d) connecting said fixation unit to the jaw and/or teeth of said patient;
e) attaching said tool to said interfacing unit such that movement of said tool by said practitioner causes a corresponding movement of said interfacing unit relative to said fixation unit;
f) activating said tool while moving said tool according to said surgery or treatment path designed in step b;
g) sensing, by means of sensors placed at different locations on said guidance device, the location and the orientation of said of said interfacing unit and attached tool relative to said fixation unit and transferring signals indicative of said location and orientation to a transmitter;
h) transmitting said location and orientation signals to a receiver that is a part of an analyzer unit;
i) activating a computing device in said analyzer unit, said computing device comprising software adapted to determine said instantaneous location and orientation of said tool based on said signals received by said receiver; and
j) activating a display device in said analyzer unit to display information relating to said location and orientation of said tool to said practitioner.

8. The method of claim 7, wherein steps f to j are carried out simultaneously in real-time.

9. The method of claim 7 comprising an introductory step of inserting one or more positioning elements in a jaw of the patient.

10. The method of claim 9 wherein at least one of the positioning elements is adapted to connect a fixation unit to a jaw and said method comprises the step of connecting a fixation unit to the elements.
